(19) United States
(12) Patent Application Publication
(10) Pub. No.: US 2026/0183149 A1
(43) Pub. Date: Jul. 2, 2026

(54) BALLOON TUNABLE EAR PLUG

(71) Applicant: Restorear Devices, LLC, Bozeman, MT (US)

(72) Inventors: Curtis S. King, Bozeman, MT (US); Suhrud M. Rajguru, Coral Gables, FL (US)

(21) Appl. No.: 19/438,650

(22) Filed: Jan. 1, 2026

Related U.S. Application Data
(60) Provisional application No. 63/741,270, filed on Jan. 2, 2025.

(51) Int. Cl. A61F 11/10

(52) U.S. Cl. CPC A61F 11/10

(57) ABSTRACT

A multi-lumen conduit carrying an inflatable balloon near a distal conduit end. The balloon being in fluid communication with at least one lumen to permit inflation to fit an exterior balloon surface in complete circumferential registration inside an ear canal. An inflated balloon may compress all lumen of the conduit to form a blocking ear plug. A control assembly may be attached to the conduit to provide for balloon inflation, thermal control of inflation fluid, one- or two-way auditory communication through the conduit, and/or wired or wireless communication to a remote receiver.

### BACKGROUND OF THE INVENTION

Related Applications: This application claims the benefit under 35 U.S.C. 119(e) of the filing date of Provisional Application Serial No. 63/741,270, filed January 2, 2025, for "BALLOON TUNABLE EAR PLUG", the disclosure of which is incorporated by reference as though set forth herein in its entirety.

The Field of the Invention: The present invention relates to devices adapted to fit in registration at a fixed position inside a conduit. In particular, preferred embodiments are adapted to provide a plug inside an auditory canal of a mammal.

Related Art: Ear plugs are commonly used to reduce acoustic energy applied to an ear drum, i.e., to reduce noise perceived by a wearer of the ear plugs. There are numerous challenges with efficacy of conventional ear plugs. 3D geometry of the mammalian ear canal is complex. Design of effective earplugs relies entirely on a reliable auditory seal between the skin in the ear canal and the ear plug device. Unsealed areas around an ear plug compromise noise suppression capability of the ear plug.

The most common ear plug, the expanding foam version, has poor efficacy due to the inability to establish a good auditory seal. If not installed properly, foam plugs are mostly ineffective. Molded ear plugs offer significant benefits and a much more reliable seal in the ear canal, but these devices are entirely custom made and costly. There exists a need in the art for an improved, universal fit ear plug that can create an acoustically robust seal in the ear canal, and that will fit any human ear canal, or the ear canal of an animal, such as a dog.

### BRIEF SUMMARY

The invention may be embodied to provide a device and method for selectively obstructing a conduit passage. A currently preferred device includes a multi-lumen conduit having a length extending between a proximal end and a distal end. A workable conduit includes a first lumen communicating along a length of the conduit to a first opening disposed at the distal end, and a second lumen communicating along a length of the conduit to a second opening disposed spaced apart from the distal end. A resilient and generally cylindrical balloon is associated with the distal end of the conduit. Opposite ends of the balloon are generally affixed to an exterior surface of the conduit and spaced apart on opposite sides of the second opening such that fluid passing distally through the second lumen will inflate the balloon.

In certain cases, an embodiment may include a third lumen communicating along a length of the conduit to a third opening disposed spaced apart from the distal end and between opposite affixed ends of the balloon, the third lumen to provide a drain path to evacuate fluid in a proximal direction from the balloon.

A preferred embodiment includes a control assembly that is associable with the proximal end of the conduit. An exemplary control assembly is configured and arranged in harmony with the conduit to provide an active hearing restriction device for controllable acoustic communication through the sound port. A control assembly may be configured and arranged in harmony with the conduit to provide an active communication device with controllable acoustic communication through the sound port.

An exemplary control assembly may include a speaker disposed for audible broadcast in a distal direction through the first lumen. A control assembly can include a microphone disposed to receive proximally-directed acoustic energy from the conduit. A control assembly may include a portion of a wireless communication assembly to permit wireless communication to a cooperating remote device. One control assembly includes an on-board power source. Sometimes, the on-board power source is configured for untethered operation to power the control assembly. The control assembly can be configured to permit autonomous operation of the apparatus to regulate transmission of acoustic energy through the conduit toward the first opening.

A preferred control assembly includes a fluid pump configured to communicate with the second lumen. Sometimes, the conduit and balloon are configured and arranged in harmony to permit inflation of the balloon to obstruct the first lumen.

Preferably, the conduit includes a safety element configured to resist infliction of undesired damage to a wearer due to improper installation of the distal end of the conduit into an ear canal. One workable safety element provides a surface that structurally interferes with an entrance to the ear canal. An exemplary safety element can include a blocking element anchored to the conduit and spaced apart from the distal end. A safety element may have a compressible portion configured to resist imparting damage during inadvertent direct contact with internal structure associated with an ear canal.

A preferred embodiment includes a multi-lumen conduit having a length extending between a proximal end and a distal end. The conduit includes a first lumen communicating along a length of the conduit to a first opening disposed at the distal end. A second lumen communicates along a length of the conduit to a second opening disposed spaced apart from the distal end. A resilient and generally cylindrical balloon is associated with the distal end of the conduit, opposite ends of the balloon being affixed to an exterior surface of the conduit and spaced apart on opposite sides of the second opening such that fluid passing distally through the second lumen will inflate the balloon. A control assembly is affixed to the proximal end of the conduit, the control assembly to permit inflation of the balloon. Sometimes, the control assembly permits untethered operation of the assembly. Desirably the conduit and balloon are configured and arranged in harmony such that balloon inflation to a first pressure is operable to maintain the distal end of the conduit in seated registration inside an ear canal. In certain cases, further inflation of the balloon to a second pressure is operable to obstruct the first lumen.

### BRIEF DESCRIPTION OF THE DRAWINGS

In the drawings, which illustrate what are currently regarded as the best modes for carrying out the invention:

FIG. 1 is an isometric view in elevation looking toward a distal end portion of a device that is constructed according to certain principles of the instant invention;

FIG. 2 is a side view in elevation of the embodiment in FIG. 1;

FIG. 3 is a front view of the distal end of the embodiment in FIG. 1;

FIG. 4 is a close-up view of the cross-section indicated by section 4-4 in FIG. 2, and looking in the direction of the arrows;

FIG. 4A is a close-up view of an alternative cross-section from the perspective of section 4A-4A in FIG. 2, and looking in the direction of the arrows;

FIG. 4B is a close-up view of an alternative cross-section from the perspective of section 4B-4B in FIG. 2, and looking in the direction of the arrows;

FIG. 5 is a right side view of a portion of the conduit in FIG. 6, with a broken-out section of the balloon to permit observation of a fluid port;

FIG. 6 is a cross-section through a currently preferred conduit, looking toward its distal end;

FIG. 7 is a left side view of a portion of the conduit in FIG. 8, with a broken-out section of the balloon to permit observation of a fluid port;

FIG. 8 is a cross-section through a currently conduit, looking toward its distal end;

FIG. 9 is an isometric view from above, looking proximally toward the distal end of the conduit in FIG. 1, and with the balloon inflated;

FIG. 10 is a side view of the conduit in FIG. 9;

FIG. 11 is a rear view in elevation of the conduit in FIG. 9;

FIG. 12 is a cross-section view of a portion of a human head with an embodiment partially installed in the ear canal;

FIG. 13 is a rear view taken at a cross-section of the conduit, looking in the distal direction toward a deflated balloon element;

FIG. 14 is a view similar to that in FIG. 12, with the balloon now inflated;

FIG. 15 is a close-up cross-section taken through the installed and inflated balloon element in FIG. 14;

FIG. 16 is a schematic illustrating general layout of certain elements of a preferred embodiment adapted for use in an ear-protection configuration;

FIG. 17 is a schematic illustrating general layout of certain elements of a preferred embodiment adapted for use in an ear-therapy configuration;

FIG. 18 is a view similar to that in FIG. 9, of an alternative embodiment having first and second proximally connected tubular elements disposed in communication with respective lumen of a preferred conduit, and an auxiliary electronics module disposed for communication through the sound port;

FIG. 19 is a side view of the embodiment in FIG. 18; and

FIG. 20 is a cross-section view taken through section 20-20 in FIG. 19.

### DETAILED DESCRIPTION OF THE ILLUSTRATED EMBODIMENTS

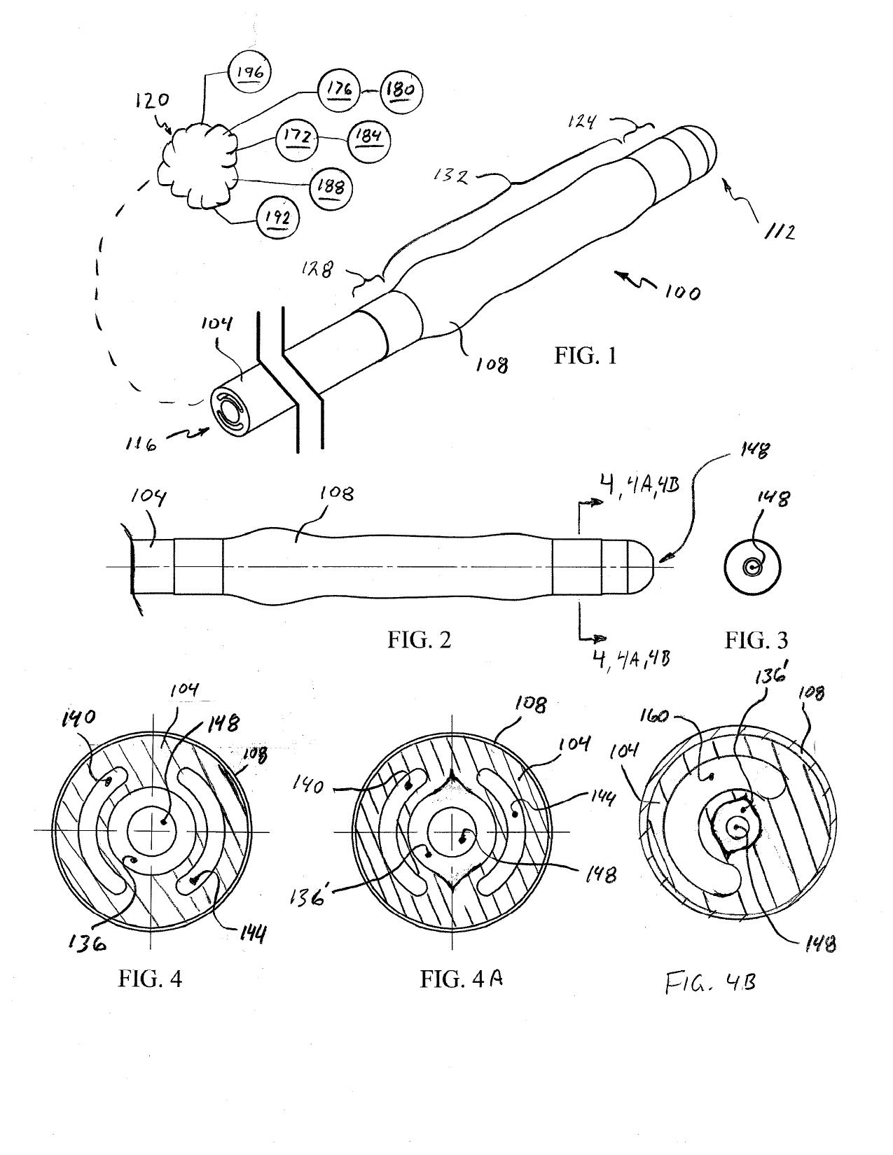

The present invention provides an apparatus and method for selectively obstructing a conduit passage. A currently preferred embodiment according to certain principles of the invention is indicated generally at 100. As illustrated in FIG. 1, an embodiment 100 may include a multi-lumen conduit 104. Conduit 104 is somewhat similar to a urinary catheter and carries a balloon 108 associated with distal end 112. The proximal end 116 of conduit 104 is typically associated with a control assembly, generally indicated at 120. A workable conduit 104 may have any desired operable length between its proximal end 116 and distal end 112.

A workable balloon 108 may be formed from a tubular length of an elastomeric membrane material. As illustrated, a distal portion 124 of balloon 108 may be anchored around a circumference to the exterior surface of the conduit 104. A proximal portion 128 may similarly be affixed to the conduit 104. Opposite ends of the intervening portion 132 are thereby sealed to the conduit to resist escape of fluid that is introduced inside the balloon 108.

With reference to FIG. 4, a currently preferred conduit 104 includes a central lumen 136, an inflation lumen 140, and a deflation lumen 144. Central lumen 136 communicates through conduit 104 and may permit distal fluid communication through distal exit aperture 148. Aperture 148 may be made reference to as a sound port, because sound may be transmitted there-through to the ear drum.

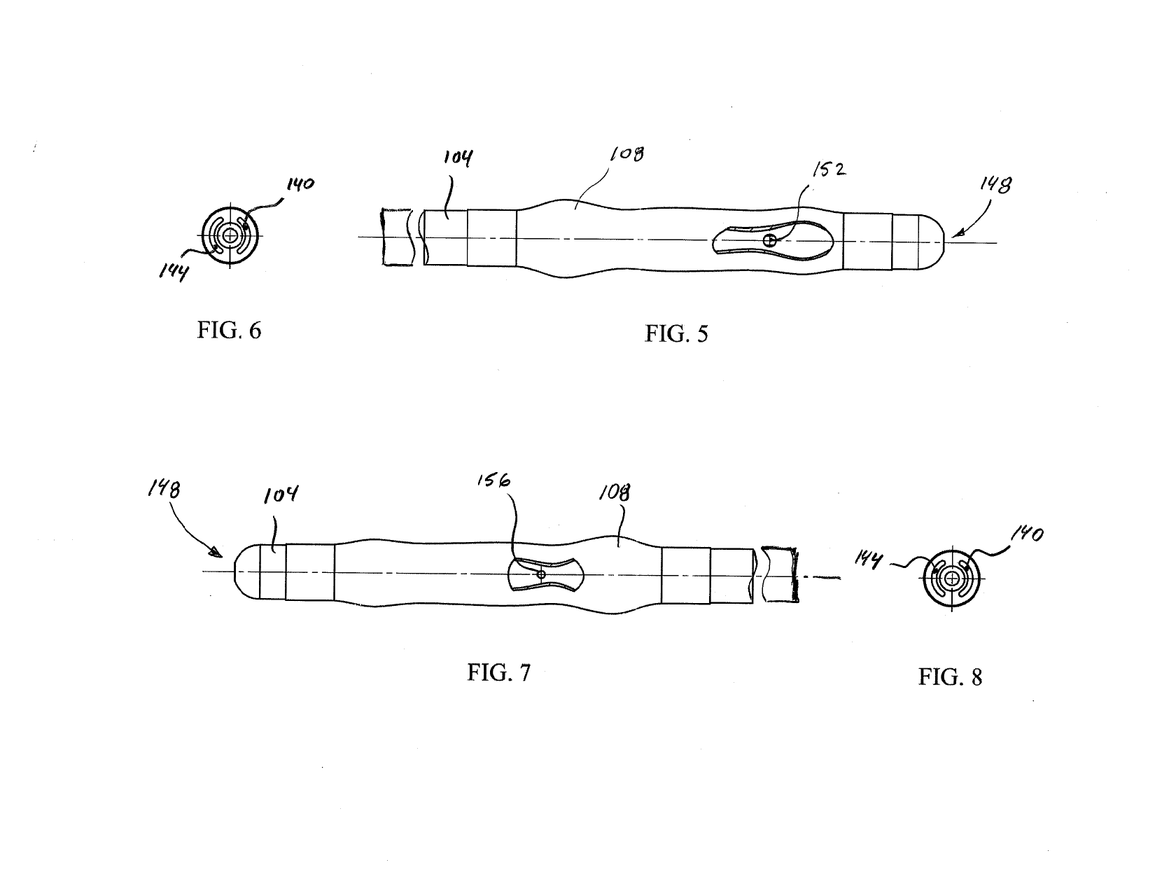

In FIG. 5, an inflation aperture 152 may be seen through a redacted portion of balloon 108. Inflation aperture 152 may communicate fluid from inflation lumen 140 to the interior of the balloon 108. Similarly, deflation aperture 156 may be seen in FIG. 7 through a redacted portion of balloon 108. Deflation aperture 156 may communicate fluid proximally from the interior of the balloon 108 through deflation lumen 144.

For purpose of this disclosure, the term “fluid” is intended to broadly encompass liquids and gasses. It is within contemplation that thermally-regulated fluid may circulate through a balloon 108 that is associated with at least two lumen of a conduit 104. A control assembly 120 may include means to regulate a temperature of circulating fluid. Therefore, an embodiment may be employed to impart thermal therapy to a wearer of a device 100.

Note that FIG. 4A illustrates an exemplary first lumen 136’ particularly shaped in cross-section to facilitate a workable blocking collapse of the cross-section under pressure exerted by the balloon 108, and more particularly by fluid pressure in lumen 160. With reference now to FIG. 4B, an alternative conduit 104 may include only two lumen. In that case, a first lumen 136” may be arranged similarly to the embodiment in FIG. 4A. A second lumen 160 can be provided to permit bidirectional fluid flow toward and away from the balloon 108. In this case, a single aperture may fluidly communicate between the lumen 160 and the inside of a balloon 108.

With reference again to FIG. 1, a control assembly 120 may nonexclusively include a fluid pump 172 to urge fluid flow, a speaker arrangement 176 to introduce sound into a conduit 104, a microphone arrangement 180 to receive sound from the conduit 104, one or more valve(s) for fluid flow control through the conduit 104, an on-board power source 188, a wired or wireless communication module 192 to convey data to a remote location, and a programmable computer or logic assembly for total or partial autonomous operation of the embodiment 100.

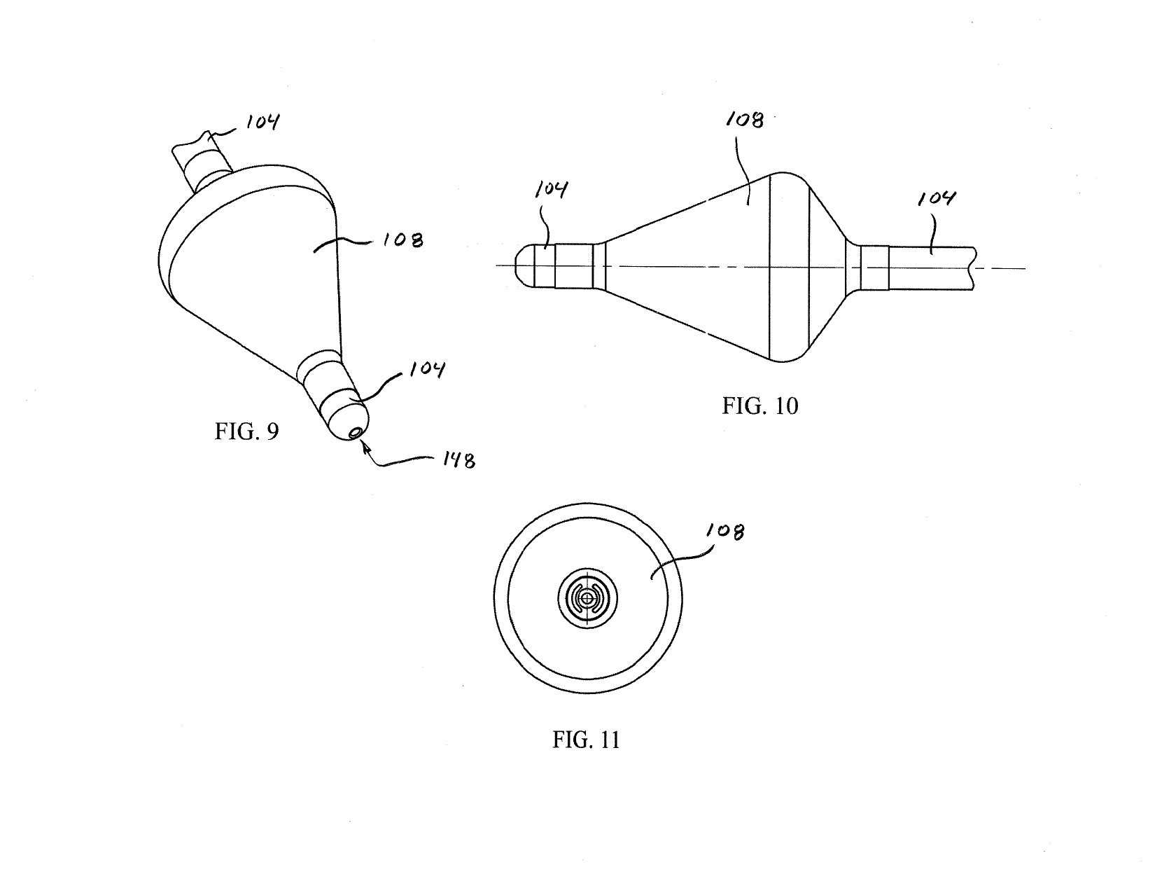

FIGS. 9 through 11 illustrate a workable shape for an inflated balloon 108. Other shapes are workable, nonexclusively including spherical or resembling a hot dog.

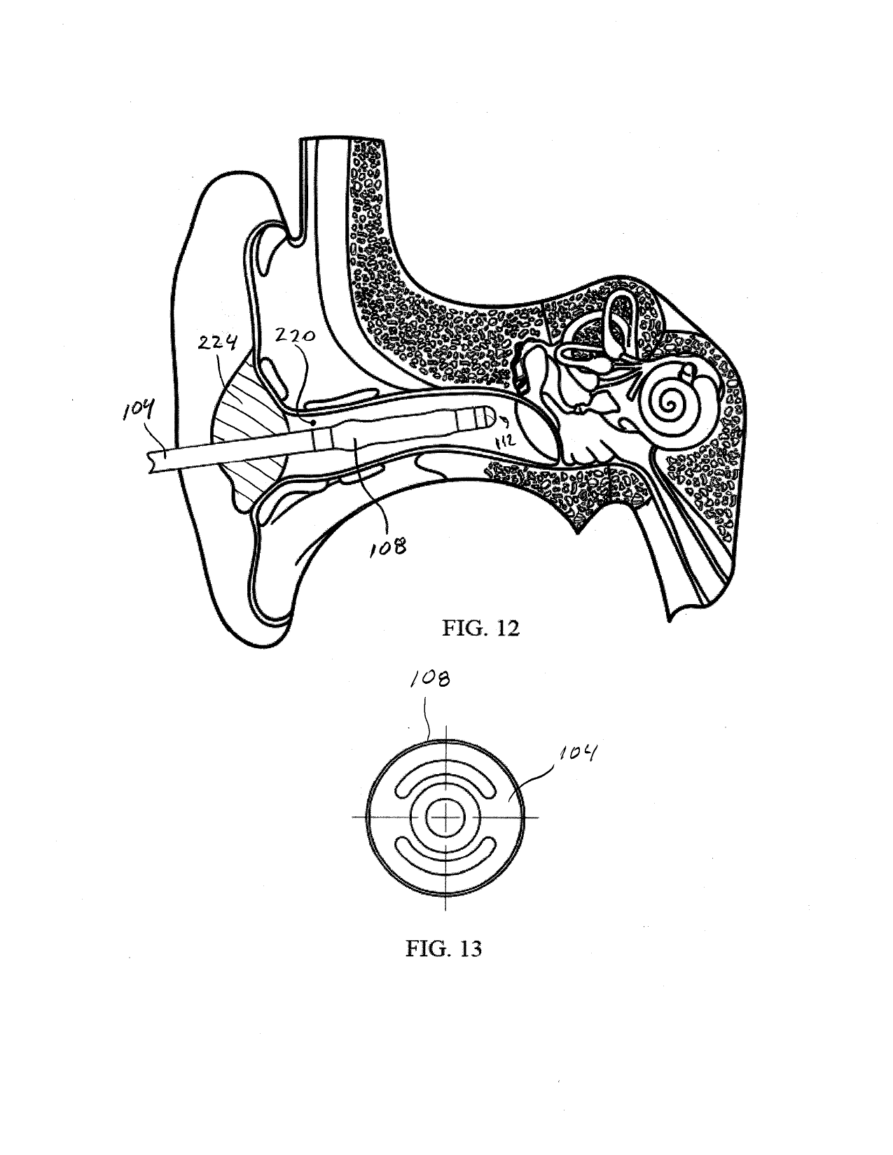

FIGS. 12 and 13 represent a distal portion of an unpressurized conduit 104 being installed in registration inside an ear canal 220. Preferably, a conduit 104 is associated with a safety element configured to resist infliction of undesired damage to a wearer due to improper installation of the distal end 112 of the conduit into an ear canal 220. As illustrated, a workable safety element 224 may be embodied to include a surface that structurally interferes with an entrance to the ear canal 220. Safety element 224 includes a blocking element (illustrated in cross-section) anchored to the conduit 104 and spaced apart from the distal end 112. The cross-section in FIG. 13 reflects a cross-section that is unpressurized.

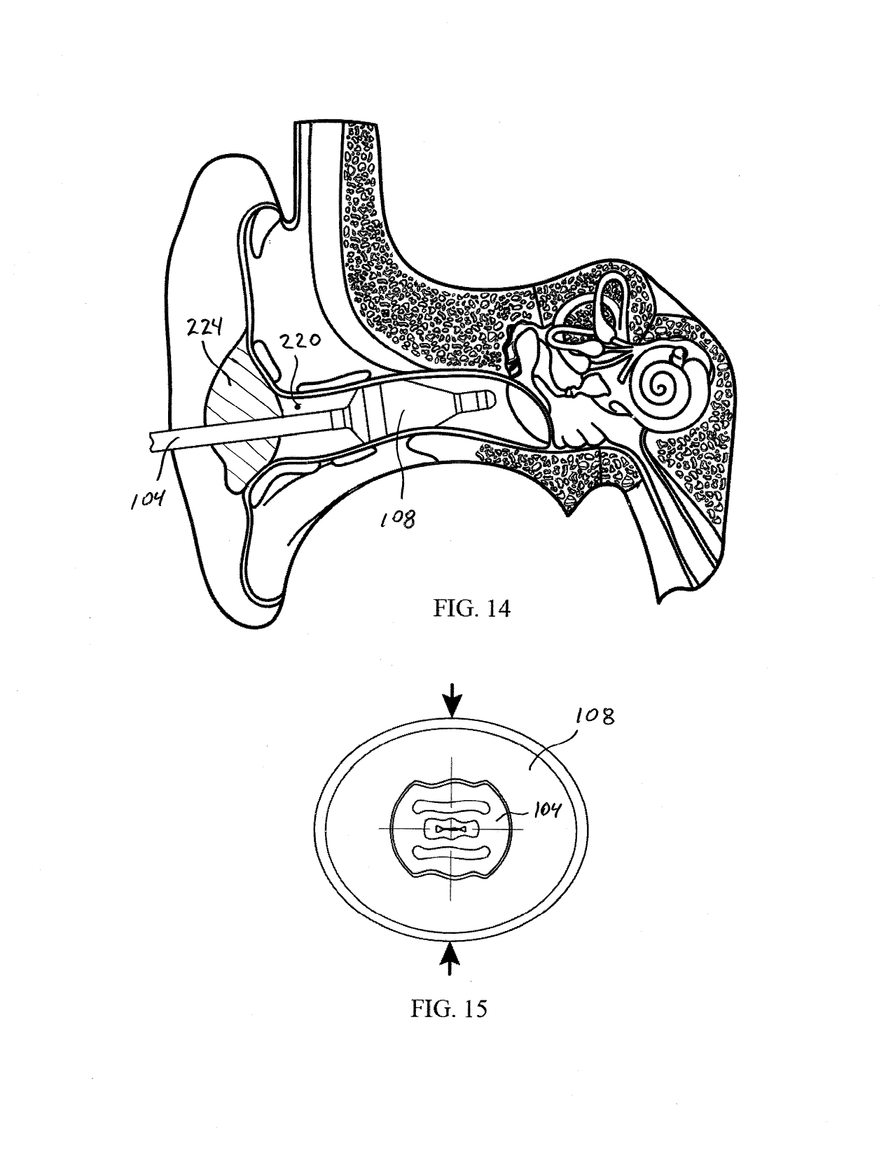

FIGS. 14 and 15 represent the distal portion of a conduit 104 with balloon 108 being seated in extra-pressurized registration inside an ear canal 220. The collapsed cross-section in FIG. 15 represents reduction in cross-section of central sound-carrying lumen 136 due to sufficient pressure in the balloon 108.

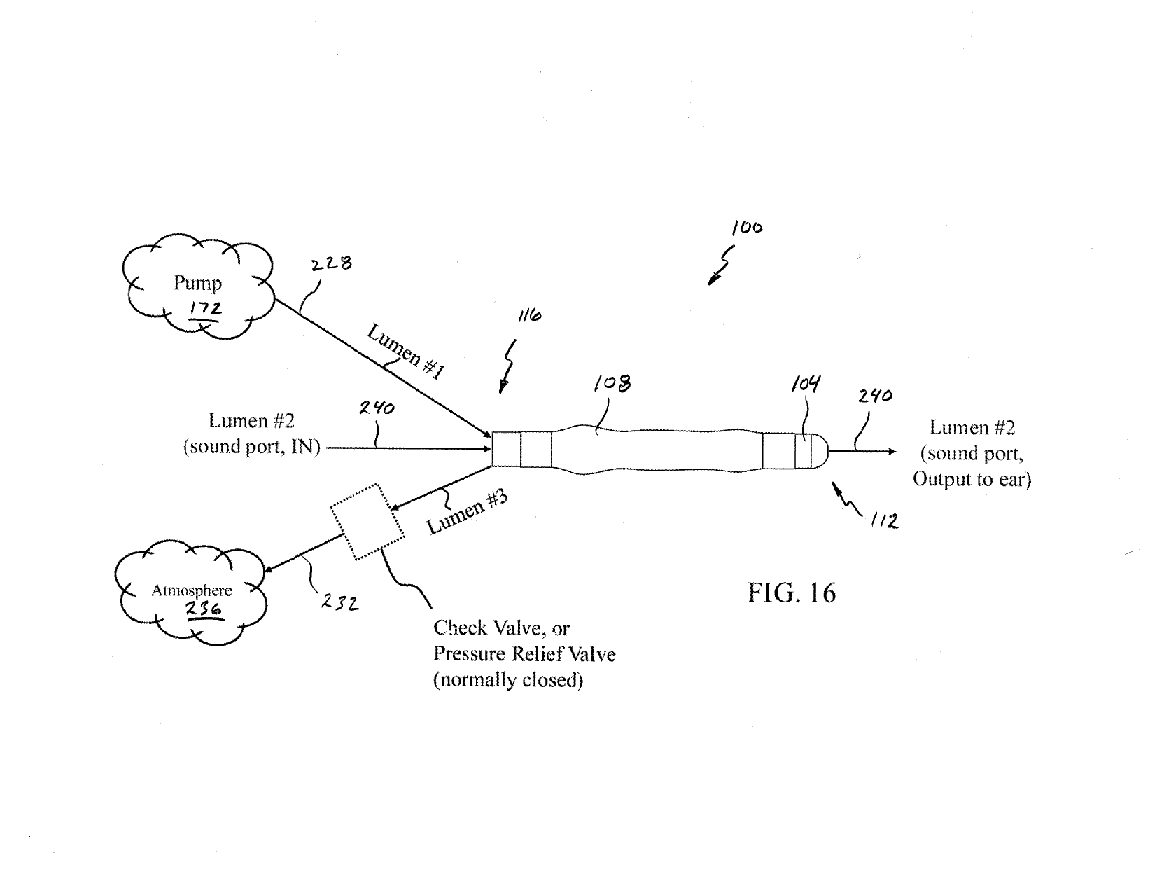

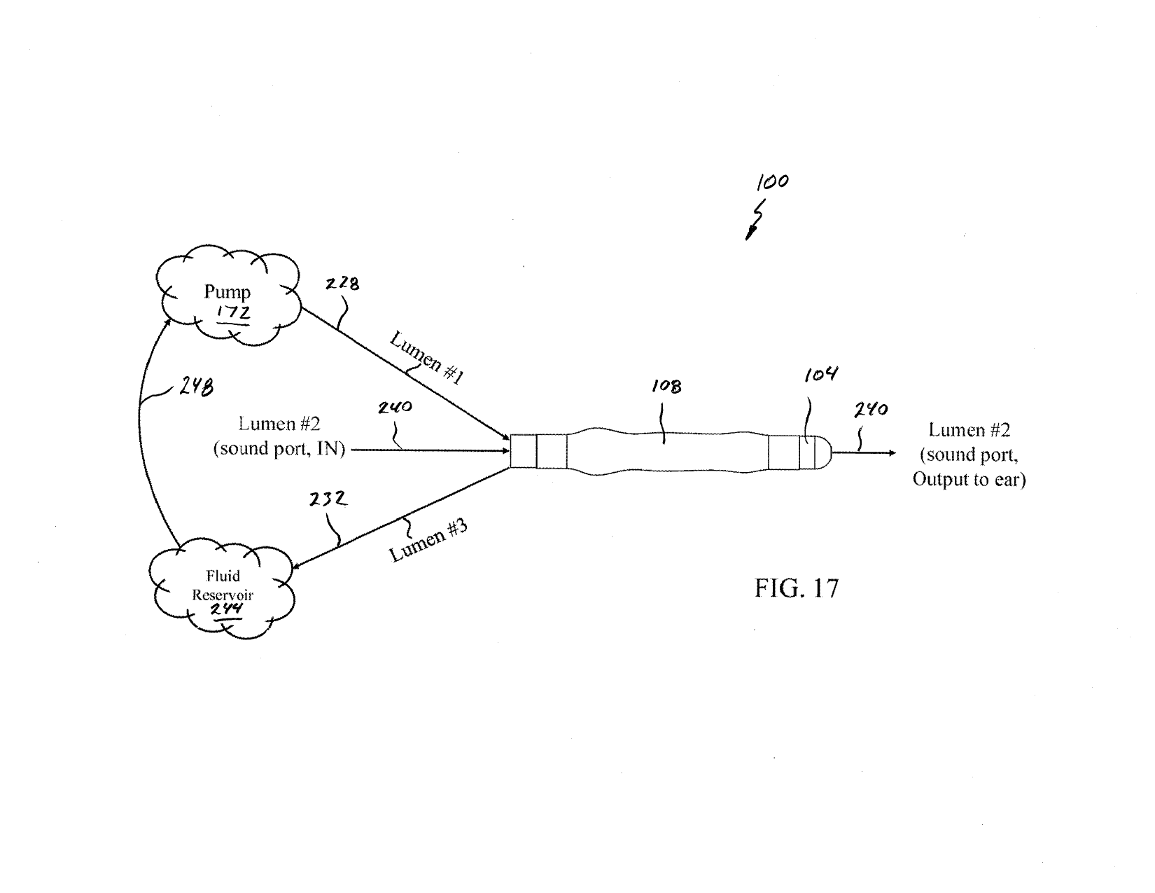

FIG. 16 illustrates an exemplary arrangement for use of an embodiment 100. A pump 172 may be configured to provide a fluid stream or input 228 to a lumen 140. Introduced fluid may pass through balloon 108 and exit through a lumen 144 as an exit stream 232 to atmosphere 236. A sonic input 240 may be applied as desired to lumen 136 to provide an output directed toward an ear drum of a wearer of the embodiment 100. In an alternative arrangement illustrated in FIG. 17, output stream 232 may be captured in a reservoir 244. The captured fluid can then be modified (e.g,. returned to a desired temperature), as desired. A recirculation stream 248 may complete a recirculation loop for the reconstituted treatment fluid.

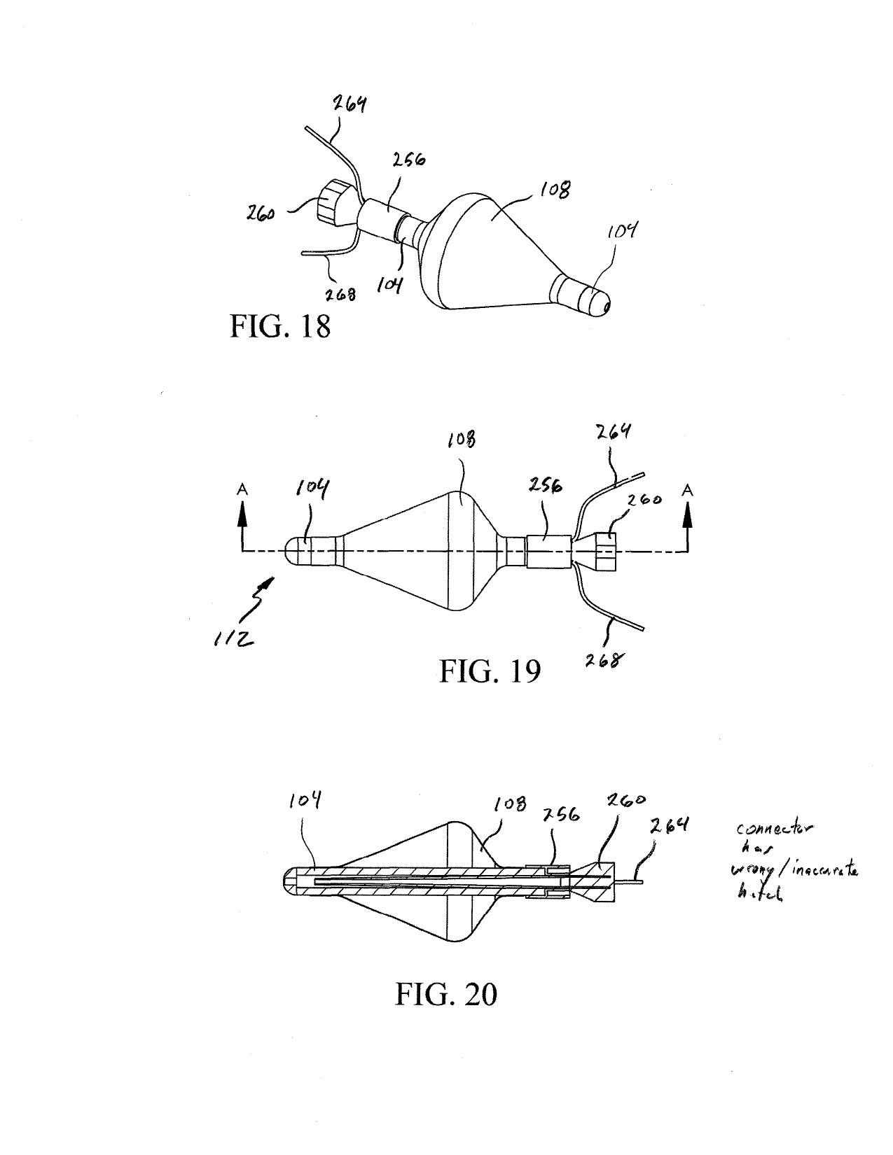

FIGS. 18-20 illustrate an alternative plumbing arrangement that can be employed in an embodiment 100. A coupling 256 secures a fitting 260 to conduit 104. The fitting 260 provides convenient means to attach flexible tubes 264 and 268 for communication with lumens 140, 144 of conduit 104. Proximal ends of the flexible tubes may facilitate placing the conduit 104 in fluid communication to ancillary equipment.

It is within contemplation to include an active hearing and communication device for acoustic communication with the sound port. An exemplary embodiment may include a speaker 176 and a microphone 180, and could potentially be powered by either an on-board or an external power source 188. An embodiment may also include or otherwise be coupled to a wireless communication or radio module 192 (e.g., Bluetooth, Wi-Fi, VHF, UHF, satellite, etc) for electronic communication between the wearer and another party, or date collection.

In one workable case, a speaker and microphone can be part of a communications module that can be associated, either permanently or removably, for communication through the sound port of the conduit 104’s shaft.

During use of an embodiment 100 as hearing protection, a balloon 108 may inflate responsive to a threshold stimulus or command signal. Balloon inflation may be controlled by an assembly 120 to provide a desired degree of sound transmission, depending on the nature of the stimulus or command signal.

An inflated balloon 108 desirably first creates a robust ear seal to resist passage of sound toward an eardrum and external to the internally-disposed sound port lumen 136 of the conduit 104. At this first condition, communication to or from an ear structure through a sound port lumen 136 may still be broadcast or received via speaker and microphone contained within a communication module associated with the sound port lumen 136.

Given an operable command signal, or an above-threshold stimulus, the pressure in balloon 108 may be automatically increased to pinch closed the sound port lumen 136, and thereby provide hearing protection from, e.g., a loud sonic event.

It is within contemplation that an embodiment 100 may be configured for portable use untethered to other device(s). For example, lumen 140 and lumen 144 of e.g., the embodiment illustrated in FIG. 16, may each communicate to a workable check valve, pressure-relief valve, pump, or other fluid control device, to permit untethered use of, and/or control of pressure in, the balloon. Therefore, an embodiment 100 may be installed and inflated to resist entrance of ambient noise into an ear canal; basically for use as a portable ear plug to resist perception of undesired ambient noise.

Certain embodiments 100 may not only provide hearing protection, but may also permit broadcast of audible commands or information to a wearer of an inflated embodiment. For example, a tethered or untethered embodiment 100 may broadcast an acoustic signal to a wearer while reducing ambient noise perceivable by the wearer. Further, an embodiment 100 may also (or alternatively) be useful in a variety of diagnostic and therapeutic applications inside the ear. For example, circulating liquid may be used to pressurize the balloon 108. The temperature of that liquid may be externally controlled, such that the balloon 108, when inflated, could deliver via direct thermal conduction, hypothermic therapy to sensory structures of the inner ear. An acoustic or auditory probe, potentially similar to a commercially available probe from Intelligent Hearing Systems having a website address of world wide web //ihsys.info/site/en/, may be used for data acquisition, emission of sonic signals or commands, and/or therapy.

From a diagnostic perspective, delivery of sound via the speaker and recording sound via the microphone permit a clinician to perform otoacoustic emissions, and functional hearing testing. Other protective, diagnostic, communication, and therapeutic applications are contemplated.

While aspects of the invention have been described in particular with reference to certain illustrated embodiments, such is not intended to limit the scope of the invention. The present invention may be embodied in other specific forms without departing from its spirit or essential characteristics. For one example, one or more element may be extracted from one described or illustrated embodiment and used separately or in combination with one or more element extracted from one or more other described or illustrated embodiment(s), or in combination with other known structure. The described embodiments are to be considered as illustrative and not restrictive. Obvious changes within the capability of one of ordinary skill are encompassed within the present invention. All changes which come within the meaning and range of equivalency of the claims are to be embraced within their scope.

What is claimed is:

1. An apparatus, comprising:
a multi-lumen conduit having a length extending between a proximal end and a distal end, the conduit comprising:
a first lumen communicating along a length of the conduit to a first opening disposed at the distal end; and
a second lumen communicating along a length of the conduit to a second opening disposed spaced apart from the distal end;
a resilient and generally cylindrical balloon associated with the distal end of the conduit, opposite ends of the balloon being affixed to an exterior surface of the conduit and spaced apart on opposite sides of the second opening such that fluid passing distally through the second lumen will inflate the balloon; and
a control assembly associable with the proximal end of the conduit.

2. The apparatus according to claim 1, further comprising:
a third lumen communicating along a length of the conduit to a third opening disposed spaced apart from the distal end and between opposite affixed ends of the balloon, the third lumen to provide a drain path to evacuate fluid in a proximal direction from the balloon.

3. The apparatus according to claim 1, wherein;
the conduit and balloon are configured and arranged in harmony to permit inflation of the balloon to obstruct the first lumen.

4. The apparatus according to claim 1, wherein:
the control assembly is configured and arranged in harmony with the conduit to provide an active hearing restriction device for controllable acoustic communication through the sound port.

5. The apparatus according to claim 1, wherein:
the control assembly is configured and arranged in harmony with the conduit to provide an active communication device comprising controllable acoustic communication through the sound port.

6. The apparatus according to claim 5, wherein:
the control assembly comprises a speaker disposed for audible broadcast in a distal direction through the first lumen.

7. The apparatus according to claim 5, wherein:
the control assembly comprises a microphone disposed to receive proximally-directed acoustic energy from the conduit.

8. The apparatus according to claim 1, wherein:
the control assembly comprises a portion of a wireless communication assembly to permit wireless communication to a cooperating remote device.

9. The apparatus according to claim 1, wherein:
the control assembly comprises an on-board power source.

10. The apparatus according to claim 9, wherein:
the on-board power source is configured for untethered operation to power the control assembly.

11. The apparatus according to claim 1, wherein:
the control assembly is configured to permit autonomous operation of the apparatus to regulate transmission of acoustic energy through the conduit toward the first opening.

12. The apparatus according to claim 1, wherein:
the control assembly comprises a fluid pump configured to communicate with the second lumen.

13. The apparatus according to claim 1, wherein:
the control assembly is operably affixed to the conduit.

14. The apparatus according to claim 1, wherein:
the conduit comprises a safety element configured to resist infliction of undesired damage to a wearer due to improper installation of a the distal end of the conduit into an ear canal.

15. The apparatus according to claim 14, wherein:
the safety element comprises a surface that structurally interferes with an entrance to the ear canal.

16. The apparatus according to claim 15, wherein:
the safety element comprises a blocking element anchored to the conduit and spaced apart from the distal end.

17. The apparatus according to claim 15, wherein:
the safety element comprises a compressible portion configured to resist imparting damage during inadvertent contact with internal structure associated with an ear canal.

18. An apparatus, comprising:
a multi-lumen conduit having a length extending between a proximal end and a distal end, the conduit comprising:
a first lumen communicating along a length of the conduit to a first opening disposed at the distal end; and
a second lumen communicating along a length of the conduit to a second opening disposed spaced apart from the distal end;
a resilient and generally cylindrical balloon associated with the distal end of the conduit, opposite ends of the balloon being affixed to an exterior surface of the conduit and spaced apart on opposite sides of the second opening such that fluid passing distally through the second lumen will inflate the balloon; wherein
the conduit and balloon are configured and arranged in harmony to permit inflation of the balloon to obstruct the first lumen.

19. The apparatus according to claim 18, further comprising:
a control assembly affixed to the proximal end of the conduit, the control assembly to permit inflation of the balloon.

20. An apparatus, comprising:
a multi-lumen conduit having a length extending between a proximal end and a distal end, the conduit comprising:
a first lumen communicating along a length of the conduit to a first opening disposed at the distal end; and
a second lumen communicating along a length of the conduit to a second opening disposed spaced apart from the distal end;
a resilient and generally cylindrical balloon associated with the distal end of the conduit, opposite ends of the balloon being affixed to an exterior surface of the conduit and spaced apart on opposite sides of the second opening such that fluid passing distally through the second lumen will inflate the balloon; and
a control assembly affixed to the proximal end of the conduit, the control assembly to permit inflation of the balloon, the control assembly to permit untethered operation of the assembly; wherein
the conduit and balloon are configured and arranged in harmony such that balloon inflation to a first pressure is operable to maintain the distal end in seated registration inside an ear canal, and further inflation of the balloon to a second pressure is operable to obstruct the first lumen.
